(19) United States
(12) Patent Application Publication
(10) Pub. No.: US 2008/0294054 A1
(43) Pub. Date: Nov. 27, 2008

(54) ULTRASOUND PROBE AND DIAGNOSTIC ULTRASOUND SYSTEM

(76) Inventors: Satoru ASAGIRI, Yokohama-shi (JP); Takeshi Miyagi, Fujisawa-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/124,463

(22) Filed: May 21, 2008

(30) Foreign Application Priority Data
May 23, 2007 (JP) 2007-136889

(51) Int. Cl. A61B 8/00

(52) U.S. Cl. 600/459

(57) ABSTRACT

An ultrasound probe is provided with a piezoelectric sensor module, which includes a plurality of piezoelectric elements arranged two-dimensionally and configured to generate an ultrasound beam so that the beam is reflected by a subject's body and to capture a resulting reflection signal, and a control circuit board that is electrically connected to the piezoelectric sensor module through a flexible substrate and transmits or receives a signal to or from the piezoelectric sensor module. The control circuit board is composed of a transmit-only circuit board, a receive-only circuit board, and a relay flexible substrate that electrically connects the transmit-only and receive-only circuit boards.

### CROSS-REFERENCE TO RELATED APPLICATIONS

This application is based upon and claims the benefit of priority from prior Japanese Patent Application No. 2007-136889, filed May 23, 2007, the entire contents of which are incorporated herein by reference.

### BACKGROUND OF THE INVENTION

1. Field of the Invention

The present invention relates to an ultrasound probe and a diagnostic ultrasound system using the ultrasound probe.

2. Description of the Related Art

Diagnostic ultrasound systems that display tomographic images of the soft tissues of living organisms by the ultrasonic pulse reflection method are frequently used as medical applications of ultrasound. Unlike other diagnostic systems, such as X-ray diagnostic systems, X-ray CT systems, MRI systems, nuclear medicine diagnostic systems, etc., the diagnostic ultrasound systems can perform real-time display.

Besides, the diagnostic ultrasound systems are small and relatively low-priced. Since they do not involve X-rays, these systems can perform repeated inspections with high safety. They are advantageous in that the ultrasound probe is only expected to be simply held against the surface of a subject's body and can be moved to the bedside with ease. Thus, the diagnostic ultrasound systems are widely used in the fields of cardiosurgery, abdominal therapy, mammary treatment, urology, gynecology, etc.

Described in Jpn. Pat. Appln. KOKAI Publication No. 2001-111192 is an electronic component constituting an ultrasound sensor that obviates the necessity of side wiring for circuit boards stacked in a laminate package structure. Described in Jpn. Pat. Appln. KOKAI Publication No. 2003-079621 is an ultrasound probe and a diagnostic ultrasound system, in which a large number of electrodes in a two-dimensional array are stably connected to a transducer and a drive circuit board.

The ultrasound probe is composed of a piezoelectric sensor module also called an acoustic element, control circuit boards, and flexible substrates. The piezoelectric sensor module is provided with an ultrasonic transmit-receive element. Ultrasound signals are transferred between the ultrasonic transmit-receive element and the control circuit boards through the flexible substrates.

The control circuit boards and the flexible substrates may be electrically connected to one another with use of an anisotropic conductive film (ACF), anisotropic conductive paste, solder, conductive adhesive agent, nano-paste, or connector part. In general, a large number of signal lines corresponding to 600 or more channels are arranged two-dimensionally, so that the circuit boards and the flexible substrates are connected with the aid of the anisotropic conductive film.

In recent years, there is a tendency toward the use of multichannel systems to obtain three-dimensional images, thus requiring a high-accuracy connection technique. Since the control circuit boards are increased in area and multilayered, moreover, the ultrasound probe is inevitably larger and heavier. Since the ultrasound probe is held in the operator's hand when it is operated, in particular, the increased size and weight reduce operating efficiency and soon tire the operator.

### BRIEF SUMMARY OF THE INVENTION

In order to achieve the above object, an ultrasound probe according to the present invention comprises: a piezoelectric sensor module which generates an ultrasound beam and captures a reflection signal from a subject's body to which the ultrasound beam is applied; a transmit-only circuit board, which is electrically connected to the piezoelectric sensor module through a flexible substrate and transmit a signal to the piezoelectric sensor module; a receive-only circuit board, which is electrically connected to the piezoelectric sensor module through a flexible substrate and receive a signal from the piezoelectric sensor module; and a relay flexible substrate which electrically connects the transmit-only circuit board and the receive-only circuit board.

In order to achieve the above object, moreover, a diagnostic ultrasound system of the invention comprises: an ultrasound probe which includes a piezoelectric sensor module, which generates an ultrasound beam and captures a reflection signal from a subject's body to which the ultrasound beam is applied, a transmit-only circuit board, which is electrically connected to the piezoelectric sensor module through a flexible substrate and transmit a signal to the piezoelectric sensor module, a receive-only circuit board, which is electrically connected to the piezoelectric sensor module through a flexible substrate and receive a signal from the piezoelectric sensor module, and a relay flexible substrate which electrically connects the transmit-only circuit board and the receive-only circuit board; a data processing section which generates image data indicative of information on the subject's body based on the signal detected from the subject's body by means of the ultrasound probe; and a monitor section which displays the image data processed by the image processing section.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a configuration view showing an outline of a profile of an ultrasound probe according to an embodiment of the invention;

FIG. 2 is a block diagram schematically showing an electronic circuit of a diagnostic ultrasound system to be connected to the ultrasound probe of the embodiment;

FIG. 3 is a configuration view showing an outline of a piezoelectric sensor module constituting the ultrasound probe of the embodiment;

FIG. 4 is a configuration view showing an outline of the ultrasound probe of the embodiment;

FIG. 5A is a configuration view schematically showing a control circuit board according to the embodiment;

FIG. 5B is a configuration view schematically showing a conventional control circuit board as a comparative example for the control circuit board of the embodiment;

FIG. 6A is a configuration view schematically showing the ultrasound probe of the embodiment; and

FIG. 6B is a configuration view schematically showing a conventional ultrasound probe as a comparative example for the ultrasound probe of the embodiment.

### DETAILED DESCRIPTION OF THE INVENTION

An embodiment of the present invention will now be described with reference to the accompanying drawings.

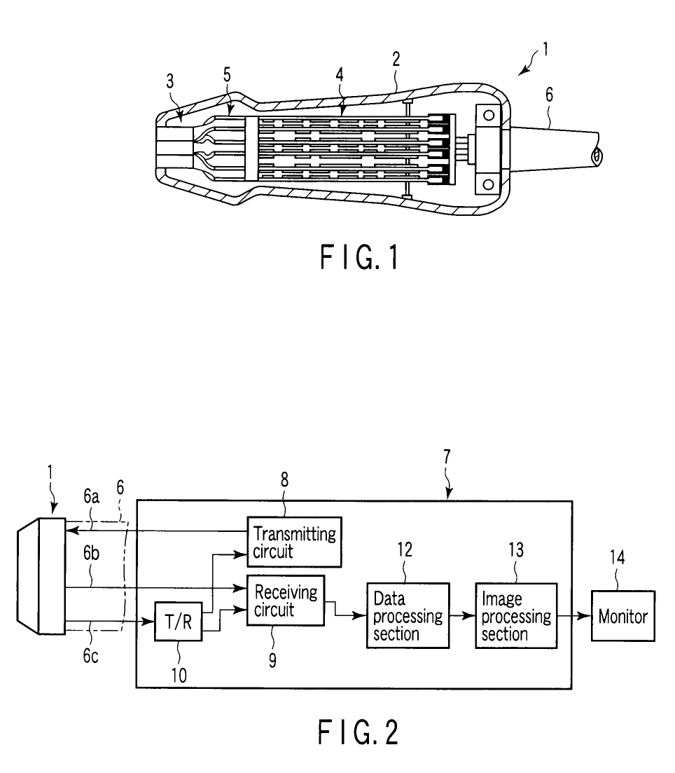

FIG. 1 is a schematic sectional view of an ultrasound probe 1.

The ultrasound probe 1 is configured so that a piezoelectric sensor module 3 is contained in the distal end portion of a hand case 2. Further, control circuit boards 4 are contained in the hand case 2 so as to occupy most of the interior of the hand case 2. Flexible substrates 5 are interposed between the sensor module 3 and the circuit boards 4 so as to connect them electrically.

Further, the ultrasound probe 1 includes a cable 6 that projects from the rear end of the hand case 2. The ultrasound probe 1, of which the hand case 2 contains the piezoelectric sensor module 3, control circuit boards 4, and flexible substrates 5, is electrically connected to a diagnostic ultrasound system 7 (mentioned later) by the cable 6.

FIG. 2 is a block diagram schematically showing an electronic circuit of the diagnostic ultrasound system 7.

The cable 6 that is connected between the ultrasound probe 1 and the diagnostic ultrasound system 7 is formed by bundling a plurality of transmit-only cables 6a, a plurality of receive-only cables 6b, and a transmit/receive cable 6c together.

The transmit-only cables 6a are connected to a transmitting circuit 8 that generates transmit pulses. The receive-only cables 6b are connected to a receiving circuit 9 that generates receiver signals mainly by digital beam forming. The transmitting circuit 8 includes circuits for two-dimensional scanning and three-dimensional scanning. The receiving circuit 9 includes circuits for two-dimensional scanning and three-dimensional scanning. The transmit/receive cable 6c is alternatively connected to the transmitting circuit 8 or the receiving circuit 9 through a transmit/receive switch 10, depending on the signal transfer mode, transmission or reception.

A data processing section 12 is connected to the receiving circuit 9 and generates image data that represents tissue-form information based on the amplitude of a receiver signal from the receiving circuit 9. Specifically, the data processing section 12 scans an ultrasound beam in a subject's body, and a resulting reflection signal is subjected to luminance modulation on a CRT as a display is presented corresponding to the scanning on the CRT. Thus, a diagnostic ultrasound image of the subject's body is displayed.

The data processing section 12 performs processing for a color flow mapping mode, thereby generating color flow mapping mode data indicative of the distribution of velocity values for blood flows and the like, power values, and distributed values. An image processing section 13 performs predetermined processing for the image data obtained in the data processing section 12. A monitor section 14 displays an image based on the image data processed by the image processing section 13.

In the diagnostic ultrasound system 7 provided with the electronic circuit arranged in this manner, the piezoelectric sensor module 3 that constitutes the ultrasound probe 1 generates an ultrasound beam and reflects it on the subject's body. Then, the system 7 captures a resulting reflection signal, thereby obtaining an ultrasound image based on a detection signal from the subject's body.

The following is a detailed description of the ultrasound probe 1.

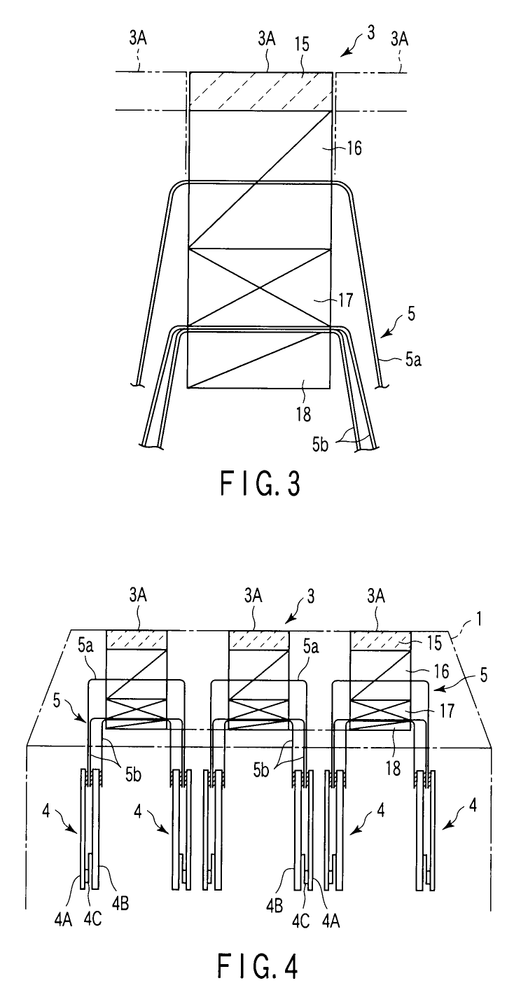

FIG. 3 is a view schematically showing a configuration of the piezoelectric sensor module 3.

The piezoelectric sensor module 3 is divided into a plurality of parts. FIG. 3 shows one divided part 3A, among others, of the sensor module 3. The sensor module 3 is constructed as a combination of several such divided module parts 3A and contained in the distal end portion of the ultrasound probe 1.

A lens portion 15 is disposed at the extreme distal end portion of each piezoelectric sensor module divided part 3A. An acoustic matching layer 16 is connected to the lens portion 15, while a transducer (piezoelectric ceramic portion) 17 is connected to the matching layer 16. A backing plate 18 is bonded to the transducer 17 to form the divided part 3A.

An acoustic lens is used for the lens portion 15, which directly contacts the subject's body to transmit or receive an ultrasound beam to or from it. The acoustic matching layer 16 is formed of a composite material that is prepared by dispersing alumina powder into epoxy resin, for example. It is subjected to two-dimensional array processing that is amenable to the transducer 17.

The transducer 17 is composed of a plurality of piezoelectric elements (ultrasound oscillators) that are two-dimensionally arranged using a piezoelectric single crystal. This piezoelectric single crystal may suitably be a single crystal of a solid solution of lead zinc niobate and lead titanate, a single crystal of a solid solution of lead magnesium niobate and lead titanate, or a single crystal of lead lithium niobate, for example.

Since the piezoelectric single crystal that forms the transducer 17 has a Curie point as low as about 180° C., moreover, it easily undergoes polarization degradation attributable to heat that is produced by soldering or array processing. Therefore, the piezoelectric single crystal is subjected to processing that causes re-polarization after array processing.

The backing plate 18 is bonded to the lower layer of the transducer 17, thereby forming an attenuation layer with a thickness of 1 to 2 mm. A rubber-based backing material is used as the material of the backing plate 18. It may, for example, be a mixture of neoprene rubber and ferrite powder or a mixture of chloroprene rubber and epoxy resin.

Further, a flexible substrate 5a for transmission is electrically connected to the acoustic matching layer 16 by predetermined means based on the use of, for example, an anisotropic conductive adhesive film. In FIG. 3, the flexible substrate 5a for transmission is shown as penetrating the acoustic matching layer 16 for the sake of simplicity.

Furthermore, a large number of electrodes are arranged on the bottom surface of the transducer 17, and they are electrically connected to a plurality of flexible substrates 5b for reception. Also for the sake of simplicity, in this case, the flexible substrates 5b for reception are shown as penetrating between the transducer 17 and the backing plate 18.

The piezoelectric sensor module divided parts 3A are contained in the ultrasound probe 1 in the manner shown in FIG. 4.

FIG. 4 is a view schematically showing component configurations in the ultrasound probe 1.

The one piezoelectric sensor module 3 is composed of three piezoelectric sensor module divided parts 3A in a bunch. Although the divided parts 3A are shown as being spaced from one another in FIG. 4, they actually are fixed in close contact with one another.

The flexible substrates 5a for transmission and the flexible substrates 5b for reception extend from the opposite side surfaces of the completed piezoelectric sensor module 3. The flexible substrates 5a and 5b extend also from between the combined piezoelectric sensor module divided parts 3A. All the flexible substrates 5a and 5b for transmission and reception are electrically connected to the control circuit boards 4.

Each control circuit board 4 is composed of a transmit-only circuit board 4A and a receive-only circuit board 4B. These circuit boards 4A and 4B are electrically connected to each other by a relay flexible substrate 4C. Thus, the relay flexible substrate 4C is also a component part of the control circuit board 4.

As shown in FIG. 4, the flexible substrates 5a for transmission are situated over the flexible substrates 5b for reception and individually connected to the control circuit boards 4 below them. In each piezoelectric sensor module divided part 3A, moreover, the flexible substrate 5a for transmission that projects from the opposite side surfaces of the divided part 3A is situated outside the flexible substrates 5b for reception.

The flexible substrate 5a for transmission is electrically connected to one side surface of the upper end portion of the transmit-only circuit board 4A with an anisotropic conductive film (ACF) therebetween. The flexible substrates 5b for reception are electrically connected to the opposite side surfaces of the upper end portion of the receive-only circuit board 4B with anisotropic conductive films therebetween.

Thus, as regards each single piezoelectric sensor module divided part 3A, the flexible substrate 5a for transmission is situated outside, and the flexible substrates 5b for reception inside. Accordingly, the transmit-only circuit board 4A that is connected to the flexible substrate 5a is situated outside, while the receive-only circuit board 4B that is connected to the flexible substrates 5b for reception is situated inside.

Since the control circuit boards 4 are disposed individually on the respective opposite sides of the three piezoelectric sensor module divided parts 3A, they are six in number, so that the transmit-only circuit boards 4A and the receive-only circuit boards 4B that constitute them are 12 in total.

The piezoelectric sensor module 3 in the ultrasound probe 1 is provided with a group of piezoelectric elements (ultrasound oscillators) that is arranged two-dimensionally and configured to generate an ultrasound beam so that the beam is reflected by the subject's body and to capture a resulting reflection signal. The control circuit boards 4 electrically connect the piezoelectric sensor module 3 and the control circuit boards 4 and transfer ultrasonic signals between the sensor module 3 and the circuit boards 4.

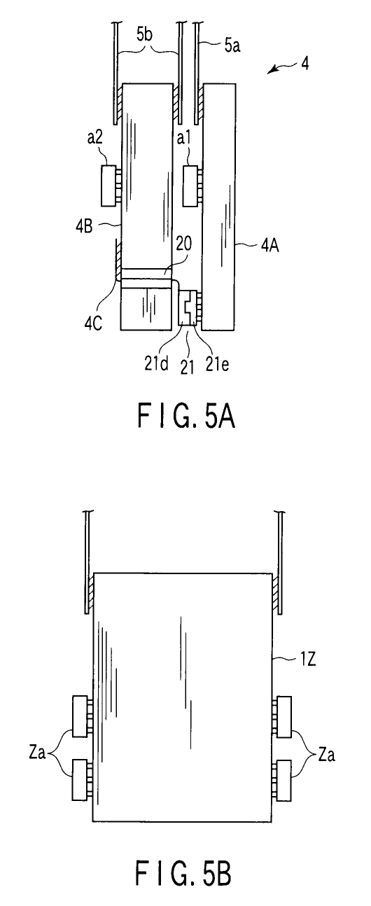

FIG. 5A is an enlarged view of the control circuit board 4 according to the present embodiment, and FIG. 5B is an enlarged view of a conventional control circuit board 1Z as a comparative example.

An ultrasound probe provided with the conventional control circuit board 1Z shown in FIG. 5B will be described first. In the group of piezoelectric elements that is two-dimensionally arranged to form the piezoelectric sensor module, one element constitutes one channel, in general. A modern conventional ultrasound probe is provided with 600 or more channels, and the piezoelectric element group is inched as the subject's body is scanned.

An electronic switch is shifted with every cycle of transmission and reception, which is performed by one control circuit board 1Z. The control circuit board 1Z is formed by laminating copper foil layers, which form signal lines, with a dielectric material therebetween. Ten or more copper foil layers are used to form the signal lines for multichannel signal processing.

Three-dimensional dynamic images are expected to be finally obtained, so that the development of multichannel systems is being advanced naturally. Thus, the control circuit board 1Z is positively increased in size and layer number, so that the operability of the ultrasound probe is reduced inevitably.

Presently, a thick conventional control circuit board 1Z with ten or more layers must be reduced in thickness. Further, electronic components Za are mounted on the opposite surfaces of the control circuit board 1Z and electrically connected to the circuit board 1Z. Thus, the thickness of the control circuit board 1Z is further increased, so that the mounting positions of the electronic components Za must also be watched.

According to the present invention, in consideration of these various conditions, the control circuit board 4 is composed of the transmit-only circuit board 4A and the receive-only circuit board 4B, which are electrically connected to each other by the relay flexible substrate 4C, as shown in FIG. 5A.

Based on various experimental results on the basic configuration described above, it was found that a signal processing capability high enough to replace the capacity of the existing control circuit board 1Z with ten or more layers can be obtained by making the transmit-only circuit board 4A and the receive-only circuit board 4B four-layered and six-layered, respectively. If the development of multichannel systems is promoted, moreover, it can be fully coped with by the use of the control circuit board 4 with the basic configuration described above.

Conventionally, furthermore, one control circuit board 1Z serves for both transmission and reception. If the number of channels is small, internal signal lines are so simple that the number of layers need not be large. In the case of a conventional advanced multichannel version, on the other hand, the internal signal lines are complicated. If a further arrangement is made, the control circuit board is given a multilayer structure including ten or more layers.

In the control circuit board 4 of the present invention, the transmit-only circuit board 4A and the receive-only circuit board 4B are completely separated for signal arrangement. Since the control circuit boards 4A and 4B are dedicated individually for transmission and reception, the signal lines in the circuit boards are simple. As mentioned before, therefore, only four layers are enough for the transmit-only circuit board 4A, and only six for the receive-only circuit board 4B.

An opening portion 20 penetrates a part of the receive-only circuit board 4B. One end portion of the relay flexible substrate 4C is connected to one side surface of the receive-only circuit board 4B through the interior of the opening portion 20. On the other surface side of the receive-only circuit board 4B, one terminal (male) 21d that constitutes a relay connector 21 is connected to the other end portion of the relay flexible substrate 4C.

The transmit-only circuit board 4A is provided with the other terminal (female) 21e that constitutes the relay connector 21. The one terminal 21d of the relay connector 21 is fitted into and connected to the other terminal 21e. The connection of the relay connector 21 is performed simultaneously with the combination of the transmit-only circuit board 4A and the receive-only circuit board 4B.

An electronic component al for transmission is mounted on one side surface of the transmit-only circuit board 4A, and an electronic component a2 for reception on one side surface of the receive-only circuit board 4B. Since the control circuit board 4 is divided into the circuit boards 4A and 4B in this manner, it is necessary only that the circuit boards 4A and 4B be mounted one-sidedly with the electronic components a1 and a2, respectively.

While the electronic component a2 for reception is mounted on the outer surface of the receive-only circuit board 4B, moreover, the electronic component a1 for transmission is mounted on a side surface of the transmit-only circuit board 4A in a gap between circuit boards 4A and 4B. Thus, the electronic component a1 is mounted only on the same side surface of the transmit-only circuit board 4A as the one to which the relay connector 21 is attached.

Since the electronic component a1 is not mounted on the outer surface of the transmit-only circuit board 4A at the least, therefore, the thickness of the control circuit board 4 that is composed of the transmit-only and receive-only circuit boards 4A and 4B can be more effectively restrained from increasing.

As mentioned before, on the other hand, the electronic components Za are mounted on the opposite surfaces of the conventional control circuit board 1Z. Therefore, the control circuit board 1Z, which is already a multilayer plate, is inevitably increased in thickness for the two electronic components a1 and a2.

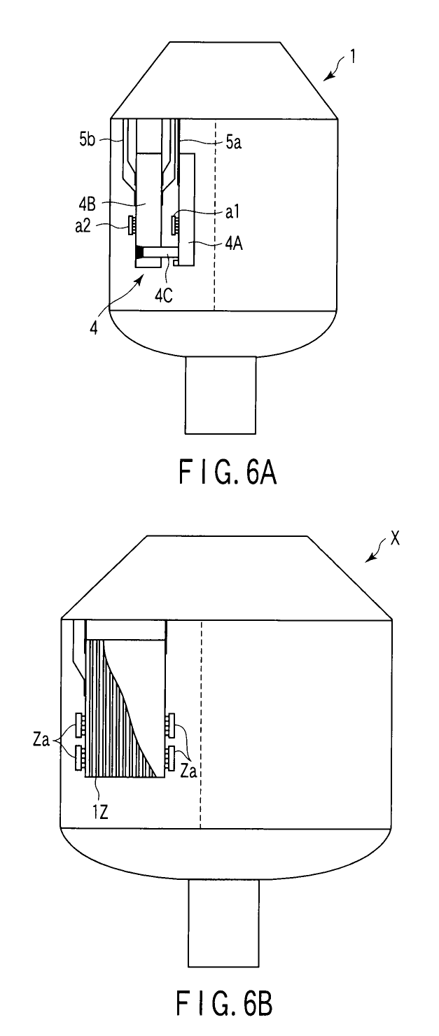

FIG. 6A is a schematic view of the ultrasound probe 1 provided with the control circuit board 4 of the present invention, and FIG. 6B is a schematic view of an ultrasound probe X with the conventional control circuit boards 1Z as a comparative example.

Conventionally, as mentioned before, each single control circuit board 1Z is a multilayer plate with ten or more layers. Thus, the ultrasound probe X, which is provided with a plurality of such control circuit boards 1Z having the electronic components Za on their opposite surfaces, is inevitably large-sized. Naturally, the ultrasound probe is increased in weight and suffers poor operability. If the number of channels increases, each control circuit board 1Z becomes thicker, so that the ultrasound probe X is further increased in size and weight.

According to the present invention, on the other hand, each control circuit board 4 is constructed by connecting the four-layer transmit-only circuit board 4A and the six-layer receive-only circuit board 4B by means of the relay flexible substrate 4C. Therefore, each control circuit board 4 is thin-walled, so that the ultrasound probe 1, which is composed of a plurality of such control circuit boards, can be reduced in size and weight. Thus, the operability of the ultrasound probe 1 is improved, so that additional development of multichannel systems can be coped with.

The present invention is not limited directly to the embodiment described above, and its components may be embodied in modified forms without departing from the scope or spirit of the invention. For example, the respective numbers of layers of the transmit-only circuit board 4A and the receive-only circuit board 4B are not limited to the above-described ones. Further, various inventions may be made by suitably combining a plurality of components described in connection with the foregoing embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound probe comprising:
a piezoelectric sensor module which generates an ultrasound beam and captures a reflection signal from a subject's body to which the ultrasound beam is applied;
a transmit-only circuit board, which is electrically connected to the piezoelectric sensor module through a flexible substrate and transmit a signal to the piezoelectric sensor module;
a receive-only circuit board, which is electrically connected to the piezoelectric sensor module through a flexible substrate and receive a signal from the piezoelectric sensor module; and
a relay flexible substrate which electrically connects the transmit-only circuit board and the receive-only circuit board.

2. An ultrasound probe according to claim 1, wherein the relay flexible substrate is provided with a relay connector which is interposed between the transmit-only circuit board and the receive-only circuit board.

3. An ultrasound probe according to claim 1, wherein the transmit-only circuit board and the receive-only circuit board are of a single-side-mounted type such that an electronic component is mounted on only one side surface of each circuit board.

4. An ultrasound probe according to claim 2, wherein the transmit-only circuit board and the receive-only circuit board are of a single-side-mounted type such that an electronic component is mounted on only one side surface of each circuit board.

5. An ultrasound probe according to claim 3, wherein the electronic component mounted on the transmit-only circuit board or the receive-only circuit board, along with the relay connector attached to the relay flexible substrate, is interposed between the transmit-only circuit board and the receive-only circuit board.

6. An ultrasound probe according to claim 4, wherein the electronic component mounted on the transmit-only circuit board or the receive-only circuit board, along with the relay connector attached to the relay flexible substrate, is interposed between the transmit-only circuit board and the receive-only circuit board.

7. A diagnostic ultrasound system comprising:
an ultrasound probe which includes a piezoelectric sensor module, which generates an ultrasound beam and captures a reflection signal from a subject's body to which the ultrasound beam is applied, a transmit-only circuit board, which is electrically connected to the piezoelectric sensor module through a flexible substrate and transmit a signal to the piezoelectric sensor module, a receive-only circuit board, which is electrically connected to the piezoelectric sensor module through a flexible substrate and receive a signal from the piezoelectric sensor module, and a relay flexible substrate which electrically connects the transmit-only circuit board and the receive-only circuit board;
a data processing section which generates image data indicative of information on the subject's body based on the signal detected from the subject's body by means of the ultrasound probe; and
a monitor section which displays the image data processed by the image processing section.

8. A diagnostic ultrasound system according to claim 7, wherein the relay flexible substrate is provided with a relay connector which is interposed between the transmit-only circuit board and the receive-only circuit board.

9. A diagnostic ultrasound system according to claim 7, wherein the transmit-only circuit board and the receive-only circuit board are of a single-side-mounted type such that an electronic component is mounted on only one side surface of each circuit board.

10. A diagnostic ultrasound system according to claim 8, wherein the transmit-only circuit board and the receive-only circuit board are of a single-side-mounted type such that an electronic component is mounted on only one side surface of each circuit board.

11. A diagnostic ultrasound system according to claim 9, wherein the electronic component mounted on the transmit-only circuit board or the receive-only circuit board, along with the relay connector attached to the relay flexible substrate, is interposed between the transmit-only circuit board and the receive-only circuit board.

12. A diagnostic ultrasound system according to claim 10, wherein the electronic component mounted on the transmit-only circuit board or the receive-only circuit board, along with the relay connector attached to the relay flexible substrate, is interposed between the transmit-only circuit board and the receive-only circuit board.
